(19) United States
(12) Patent Application Publication
(10) Pub. No.: US 2006/0210636 A1
(43) Pub. Date: Sep. 21, 2006

(54) Nanoscale core/shell particles and the production thereof

(76) Inventors: Ralph Nonninger, Saarbruecken (DE); Martin Schichtel, Dudweiler (DE)

Correspondence Address: NATH & ASSOCIATES, 112 South West Street, Alexandria, VA, 22314, US

(21) Appl. No.: 10/537,802

(22) PCT Filed: Dec. 9, 2002
(86) PCT No.: PCT/EP02/13942

§ 371 Date: Aug. 9, 2005

(51) Int. Cl. A61K 9/14; A61K 9/28; B01D 21/02

(52) U.S. Cl. 424/489; 210/702; 977/900; 977/906; 427/2.14

(57) ABSTRACT

In methods for the production of nanoscale particles having a so-called core and at least one so-called shell, either nanoscale particles of an inorganic material having a particle size of <100 nm or nanoscale particles of a magnetic material having a particle size of <100 nm are used as the core. Either at least one metal is applied by radiation-induced redox reaction or at least one inorganic material is applied by means of a pH change brought about by at least one enzyme, as a shell, in solution or in suspension, to these particles forming the core. Accordingly, core/shell particles having a core of an inorganic material or a core of a magnetic material with a shell of a metal or a shell of an inorganic material are provided. These core/shell particles are distinguished in that they are present substantially, preferably completely, as nonagglomerated particles.

The invention relates to methods for producing nanoscale particles having a so-called core and at least one so-called shell, and corresponding core/shell particles themselves.

The production of core/shell particles is very important industrially. The area of UV pigments and here especially the production of coated titanium dioxide may be singled out by way of example. As a semiconductor material, titanium dioxide has a band gap at 3.2 eV and is therefore capable of absorbing UV radiation. As an inorganic UV absorber, however, it can be used only when its surface is provided with one or more protective layers. As a result of the absorption of UV light, reactive intermediates, so-called electron-hole pairs, are formed in the crystal lattice of the titanium dioxide. Since the diffusion rates of the electrons and of the holes are substantially greater than the recombination rate, these reactive intermediates migrate to the powder surface and destroy the matrix surrounding the powders. In this case, three layers of in each case silicon dioxide, zirconium oxide and aluminum oxide are customary industrially. Another example would be the protection of electroluminescent particles by analogous protective layers from water or the application of biodegradable polymers as a temporary barrier layer. Owing to its extent and complexity, it is only possible to scratch the surface of the prior art here. However, it is important to note that the prior art relates exclusively to the coating of particles which are >100 nm. There are various reasons for this.

Many methods, for example, spray drying, are suitable only for particles having primary particle sizes of >1 μm, owing to the process technology. Other methods, such as fluidized-bed methods, CVD (chemical vapor deposition) and PVD (physical vapor deposition), operate either at high temperatures or at high relative speeds and, associated with this, high kinetic energies, both leading to coalescence of the small particles before the actual coating process. Isolated particles having particle sizes of less than 100 nm cannot be provided with a shell, a coating, in this manner.

In principle, the application of a protective shell around nanoscale particles can be effected only by means of wet chemical methods (owing to the high temperatures, physical methods would lead to agglomerates of the nanoparticles), but wet chemical methods, too, are dependent on the particles to be coated already being present in isolated form side by side before and during the coating process.

There has been no lack of attempts to coat, for example, nanoscale titanium dioxide with a protective shell analogously to pigment chemistry, but many attempts to coat nanoparticles isolated from one another individually and virtually completely with a shell have to date been unsuccessful. The reason for this is that the homogeneous particle distribution in solution present before the coating process cannot be maintained as a result of the pH change of the solution, which change is required for application of the protective shell. The particles agglomerate, and then only the agglomerates are coated.

Some of these coated, nanoscale particles have appeared on the market to date, but electron micrographs prove that these commercially available powders contain not isolated, coated particles but particle clusters which are bound to one another by an amorphous coating. Many applications cannot be carried out with the aid of these powders.

The process engineering for the coating is therefore enormously important. A pH change of the solution is frequently indispensible if the shell is to be applied by means of a wet chemical process, usually a precipitation process. What is decisive in this context is that the precipitation can be effected very homogeneously. Local dropwise addition of a base, even with stirring, is completely unsuitable for this purpose. A homogeneous pH change, for example due to the decomposition of urea or similar organic compounds, which are destroyed with the formation of ammonia, is possible. The decomposition is usually initiated by the application of an elevated temperature. A pH change initiated in this manner takes place spontaneously and generally very rapidly since an equilibrium is established very rapidly. As a result of the equilibrium being established, however, the urea is only partly decomposed, so that the pH cannot become as high (basic) as it would need to be in order to achieve complete coating. A wet chemical process step which leads to a coating around particles without a pH change can be effected only by a chemical or physical reaction taking place on the surface of the nanoparticles.

The object of the present invention is to provide certain nanoscale particles having a so-called core and at least one so-called shell, which particles are present in virtually agglomerate-free form or even completely agglomerate-free form. For this purpose, it is intended to develop corresponding methods for the production of such core-shell particles. In this way, it is intended to avoid or substantially exclude the described disadvantages of the prior art.

This object is achieved by the method having the features of claim 1 and by the method having the features of claim 4. Preferred procedures are described in the dependent claims 2 and 3, and 5 to 11. Moreover, the object is achieved by the core/shell particles having the features of claims 12, 13, 18 and 19. Preferred embodiments of these core/shell particles are described in the dependent claims 14 to 17, and 20 to 25. Preferred uses of core/shell particles according to the invention are provided by the use claims 26 and 27. The wording of all claims is hereby incorporated in this description by reference.

According to the first variant of the method according to the invention, nanoscale particles of an inorganic material having a particle size of <100 nm are used as the core for the production of nanoscale core/shell particles. At least one metal is applied as the shell, in solution or in suspension by a radiation-induced redox reaction, to these particles, which, as mentioned, form the core of the core/shell particles. In this method, the redox reaction is preferably induced by UV radiation. In particular, the metal applied as the shell may be copper or silver.

By means of this (first) method according to the invention, a metal layer is deposited by a wet chemical method on the surface of the nanoscale core particles. During the procedure, a reduction of the metal ions present in solution or in suspension takes place directly on the surface of the nanoscale particles forming the core. The inorganic materials which can be used as core particles will be explained in more detail later in the description. However, it should be emphasized at this point that in particular inorganic materials having semiconductor properties are suitable as nanoscale particles for the core. Such semiconductor materials having band gaps of, preferably, between 2 eV and 5 eV can form electron-hole pairs as a result of UV excitation. The electrons formed migrate to the surface of the core particles and reduce the metal ions present there, i.e. preferably the silver ions and/or copper ions. As a result of this process, a metal film or a metal layer is deposited on the surface of the core particles. Preferred semiconductor materials having appropriate band gaps are titanium dioxide and cerium oxide.

In a second variant of the method according to the invention for the production of nanoscale core/shell particles, nanoscale particles of a magnetic material having a particle size of <100 nm can be used as the core. At least one inorganic material as a shell is then applied to these particles, which, as mentioned, form the core of the core/shell particle, in solution or in suspension by means of a pH change which is brought about by at least one enzyme. Preferably, a pH change is effected by decomposition of urea by means of urease. In principle, all magnetic, in particular all ferromagnetic, materials can be used as magnetic material. The magnetic material is preferably iron oxide, in particular magnetite.

By means of this (second) method according to the invention, substantially two advantages are obtained. Preferably, core/shell particles whose core can be controlled by an externally applied magnetic field are obtained. This opens up completely novel fields of use for such particles. Secondly, a rapid and complete coating of individual core particles with the shell material is realized by the pH change brought about with the aid of an enzyme. This prevents the agglomeration of the particles forming the core. By the addition of enzymes, the decomposition reactions taking place during the application of the shell, such as, for example, the reaction of urea to give ammonia, can be very readily controlled. Enzymes of the urease type decompose urea completely so that a sufficiently high pH can be established. Since the enzyme reaction can be influenced by the parameters of temperature and pH, it is therefore also possible to carry out the precipitation reaction over a longer period, in particular over several hours, in order thus to establish layer thicknesses in a completely controlled manner. As already mentioned, this ensures that the nanoparticles retain their individuality even after the application of the shell, the coating.

In both method variants described, the solvent used for the preparation of the solution or of the suspension is preferably removed again after the application of the shell. The powder obtained by the removal of the solvent can then be calcined. Here, calcination is to be understood as meaning the heating of the pulverulent materials up to a certain degree of decomposition, the water of crystallization contained in the material being at least partly or preferably completely removed.

The inorganic materials used in the two method variants described can be chosen substantially freely. In particular, a nanoscale oxide, sulfide, carbide or nitride powder is involved. Nanoscale oxide powders are preferred. It is possible to use all powders which are usually used for powder sintering. Examples are (optionally hydrated) oxides, such as ZnO, CeO₂, SnO₂, Al₂O₃, CdO, SiO₂, TiO₂, In₂O₃, ZrO₂, yttrium-stabilized ZrO₂, Al₂O₃, La₂O₃, Fe₂O₃, Fe₃O₄, Cu₂O, Ta₂O₅, Nb₂O₅, V₂O₅, MoO₃ or WO₃, but also phosphates, silicates, zirconates, aluminates and stannates, sulfides, such as CdS, ZnS, PbS and Ag₂S, carbides, such as WC, CdC₂ or SiC, nitrides, such as BN, AlN, Si₃N₄ and Ti₃N₄, corresponding mixed oxides, such as metal tin oxides, e.g. indium tin oxide (ITO), antimony tin oxide, fluorine-doped tin oxide and Zn-doped Al₂O₃, luminescent pigments Y- or Eu-containing compounds, or mixed oxides having the perovskite structure, such as BaTiO₃, PbTiO₃ and lead zirconium titanate (PZT). Furthermore, mixtures of the stated powder particles can also be used.

In the case of the coating of the nanoscale inorganic material with a metal as a shell, nanoscale particles which are an oxide, hydrated oxide, chalcogenide, nitride or carbide of Si, Al, B, Zn, Zr, Cd, Ti, Ce, Sn, In, La, Fe, Cu, Ta, Nb, V, Mo or W, particularly preferably of Fe, Zr, Al, Zn, W and Ti, are preferably used as the core. Oxides are particularly preferably used. Preferred nanoscale, inorganic solid particles are aluminum oxide, zirconium oxide, titanium oxide, iron oxide, silicon carbide, tungsten carbide and silicon nitride.

In the case of the use of the nanoscale inorganic material as shell material, (optionally hydrated) oxides, such as ZnO, CeO₂, SnO₂, Al₂O₃, CdO, SiO₂, TiO₂, In₂O₃, ZrO₂, yttrium-stabilized ZrO₂, Al₂O₃, La₂O₃, Fe₂O₃, Fe₃O₄, Cu₂O, Ta₂O₅, Nb₂O₅, V₂O₅, MoO₃ or WO₃, are preferably chosen, but also corresponding mixed oxides, such as metal tin oxides, e.g. indium tin oxide (ITO), antimony tin oxide, luminescent pigments with Y- or Eu-containing compounds, or mixed oxides having the perovskite structure, such as BaTiO₃, PbTiO₃ and lead zirconium titanate (PZT).

Furthermore, the invention embraces two variants of core/shell particles having a so-called core and at least one so-called shell.

In the first variant, the core/shell particles according to the invention are defined in such a way that the core comprises nanoscale particles of an inorganic material having a particle size <100 nm and the shell is at least one metal. These core/shell particles are present substantially, preferably completely, as nonagglomerated particles.

In particular, the core/shell particles according to the invention, of this first variant, can be obtained or can be prepared by the above-defined method, in which a metal is applied as a shell to a nanoscale inorganic core material by a radiation-induced redox reaction, preferably by UV radiation.

The core/shell particles according to the invention, of the first variant (core=nanoscale inorganic material; shell=metal), can be composed of the materials already described above in relation to the (first) method according to the invention. Core/shell particles of the first variant in which the inorganic material has semiconductor properties and/or in which the inorganic material is a nanoscale oxide powder are preferred. In particular, the inorganic material which forms the core of such particles is titanium oxide (TiO₂). In particular, metals having a biocidal action, and preferably silver or copper, are used as metal which forms the shell of such particles. Accordingly, preferred core/shell particles of the first variant are composed of a titanium oxide core and a shell of silver and/or copper.

In a second variant, the core/shell particles according to the invention, having a so-called core and at least one so-called shell, are defined in such a way that the core comprises nanoscale particles of a magnetic material having a particle size of <100 nm and the shell is at least one inorganic material. In this variant, too, the core/shell particles are present substantially, preferably completely, as nonagglomerated particles.

In particular, these core/shell particles according to the invention, of the second variant, can be prepared or can be obtained by the above-described method, in which an inorganic material is applied as a shell to an nanoscale magnetic material by means of a pH change brought about by at least one enzyme.

As already described in relation to the (second) method according to the invention, all magnetic, in particular all ferromagnetic, material can in principle be used as magnetic material. In particular, the magnetic material is iron oxide, preferably magnetite. Regarding the shell materials used in this second variant, reference may likewise be made to the above description. The corresponding inorganic materials are preferably a nanoscale oxide powder, in particular titanium oxide (TiO₂). Accordingly, the core/shell particles according to the invention, of the second variant, are preferably those having a core of iron oxide, in particular magnetite, and a shell of titanium oxide.

In both variants of the core/shell particles according to the invention, the nanoscale particles which form the core (inorganic material or magnetic material) preferably have a particle size between 5 nm and 50 nm, in particular between 5 nm and 20 nm.

The core/shell particles according to the invention themselves have an (average) particle size between 5 nm and 100 nm, preferably between 10 nm and 50 nm. Within the latter range, (average) particle sizes between 20 nm and 45 nm are further preferred. Preferred layer thicknesses for the shell are between 0.1 nm and 20 nm, in particular between 1 nm and 10 nm. In the invention, preferred layer thicknesses (coating thicknesses) between 0.1 nm and 2 nm can be realized without problems.

Of course, the invention is not limited to the production and provision of core/shell particles having a core and only one shell layer. Depending on the desired application, two or more shell layers can be applied, preferably in succession, to a core material.

The core/shell particles according to the invention, of both variants, are usually present as a nanoscale powder, as obtained, for example, by the methods described, by removal of the solvent and calcination. In other preferred embodiments, the particles according to the invention are either applied to an inorganic or organic carrier or introduced into an inorganic or organic matrix. In this way, they can better display the effect required for the desired application.

Finally, the invention embraces certain preferred uses of the core/shell particles according to the invention. Thus, the core/shell particles of the first variant (core=nanoscale inorganic material; shell=metal) are particularly suitable as biocides, i.e. as substances which inhibit or completely stop the growth of bacteria. The core/shell particles according to the invention, of the second variant (core=nanoscale magnetic material; shell=inorganic material), are particularly suitable for wastewater treatment, in particular for removing heavy metals from wastewaters. It is known that, for example, titanium dioxide is suitable for separating heavy metals from bodies of water by virtue of the fact that the heavy metal cations are deposited on the titanium dioxide surface in the presence of an organic reducing agent. The problem which has been unsolved to date is, however, to remove the particles laden with heavy metals from the bodies of water again. This has been achieved to date only by means of very laborious and difficult procedures using filter units. This problem is solved by the core/shell particles according to the invention, of the second variant (e.g.: core=iron oxide; shell=titanium dioxide), since these core/shell particles can be removed from the water/wastewater by applying a magnetic field.

The features of the invention described and further features of the invention are evident from the description of examples which now follows, in combination with the claims. The individual features of the invention are realized in each case alone or in combination with another.

### EXAMPLES

### Example 1

### Silver-Coated Titanium Dioxide Nanoparticles

For the production of core/shell particles according to the invention, having a core of titanium dioxide and a shell of silver, the procedure according to the first claimed method variant is followed. There, the silver is first adsorbed in the form of ions onto the titanium dioxide surface and then reduced by electrons, which are induced by UV radiation. The layer thickness of the silver can be controlled by the concentration of the silver ions in the suspension/solution and by the intensity and duration of the UV treatment.

In the specific example, an amount of 1 g of nanoscale titanium dioxide powder (titanium dioxide P 25, Degussa, Germany) is suspended in an aqueous hydrochloric acid solution (pH=2) with continuous stirring. Silver nitrate, a readily water-soluble silver salt, is added to this suspension, the amount of silver nitrate being chosen as a function of the desired layer thickness of the silver shell layer. Thereafter, the suspension is irradiated for 10 min with a UV lamp (without filter, power between 80 and 120 watts with continuous stirring). Thereafter, the work-up of the silver-coated titanium dioxide is effected by centrifuging, washing with water or dialysis through a semipermeable membrane.

In the case of the chosen irradiation time of 10 min, the following layer thicknesses can be obtained depending on the concentration of the silver ions:

| | |
|---|---|
| 0.01 mol of silver ions | layer thickness 0.1 nm |
| 0.12 mol of silver ions | layer thickness 1 nm |
| 0.32 mol of silver ions | layer thickness 2 nm |

As mentioned at the outset, the layer thickness of the silver layer can also be varied by means of the duration of irradiation. If 1 g of titanium dioxide and a silver ion concentration of 0.12 mol are initially employed, the duration of the UV irradiation has the following effect:

| | |
|---|---|
| UV radiation for 1 min | layer thickness about 0.15 mm |
| UV radiation for 5 min | layer thickness about 0.65 nm |
| UV radiation for 10 min | layer thickness about 1 nm |

### Example 2

### Cerium Oxide-Coated Magnetite Nanoparticles

For the production of core/shell particles according to the invention, having a core of magnetite and a shell of cerium oxide, the procedure according to the second claimed method variant is followed. As a result of the controlled homogeneous pH change (decomposition of urea), the cerium contained in the dispersion is deposited as cerium oxide on the surface of the magnetite.

In the specific example, an amount of 10 g of nanoscale magnetite powder (mean size about 10 nm) is suspended in 500 ml of deionized water. A polyvinyl binder which promotes the attachment of the shell material to the core material, magnetite powder, is added to this suspension. In the present case, a binder content of 1% by weight is chosen, binder contents between 0.2% by weight and 2% by weight very generally being possible without problems. 1.9 g of cerium(III) chloride and 0.4 g of urease are then added with stirring to the suspension obtained. After a constant pH has been established, 40 g of urea are added to the suspension. The suspension is then stirred for 1 h at room temperature. The work-up of the cerium oxide-coated magnetite particles obtained is effected as in example 1.

1. A method for the production of nanoscale particles having a so-called core and at least one so-called shell, wherein nanoscale particles of an inorganic material having a particle size of <100 nm are used as the core, and at least one metal is applied as the shell to these particles forming the core, in solution or in suspension, by a radiation-induced redox reaction.

2. The method as claimed in claim 1, wherein the redox reaction is induced by UV radiation.

3. The method as claim in claim 1, wherein that the metal is copper or silver.

4. A method for the production of nanoscale particles having a so-called core and at least one so-called shell, wherein nanoscale particles of a magnetic material having a particle size of <100 nm are used as the core, and at least one inorganic material is applied as the shell to these particles forming the core, in solution or in suspension, by means of a pH change brought about by at least one enzyme.

5. The method as claimed in claim 4, wherein the pH change is brought about by decomposition of urea by means of urease.

6. The method as claimed in claim 4, wherein the magnetic material is iron oxide, preferably magnetite.

7. The method as claimed in claim 1, wherein the solvent is removed after the application of the shell and, preferably, the powder thus obtained is calcined.

8. The method as claimed in claim 1, wherein the inorganic material comprises nanoscale oxide, sulfide, carbide or nitride powders, preferably nanoscale oxide powders.

9. The method as claimed in claim 8, wherein the inorganic material has semiconductor properties.

10. The method as claimed in claim 8, wherein the inorganic material is aluminum oxide, zirconium oxide, titanium oxide, iron oxide, cerium oxide, silicon carbide or tungsten carbide.

11. The method as claimed in claim 8, wherein the inorganic material is aluminum oxide (Al₂O₃) or titanium oxide (TiO₂).

12. A core/shell particle having a so-called core and at least one so-called shell,
the core comprising nanoscale particles of an inorganic material having a particle size of <100 nm,
the shell being at least one metal, and
the core/shell particles being present substantially, preferably completely, as nonagglomerated particles.

13. A core/shell particle having a so-called core and at least one so-called shell, which can be produced by the method as claimed in claim 1.

14. The core/shell particle as claimed in claim 12, wherein the inorganic material has semiconductor properties.

15. The core/shell particle as claimed in claim 12, wherein the inorganic material is a nanoscale oxide powder.

16. The core/shell particle as claimed in claim 12, wherein the inorganic material is titanium oxide (TiO₂).

17. A core/shell particle, characterized in that the metal is silver or copper.

18. A core/shell particle having a so-called core and at least one so-called shell,
the core comprising nanoscale particles of a magnetic material having a particle size of <100 nm,
the shell being at least one inorganic material, and
the core/shell particles being present substantially, preferably completely, as nonagglomerated particles.

19. A core/shell particle having a so-called core and at least one so-called shell, which can be produced by the method as claimed in claim 4.

20. The core/shell particle as claimed in claim 18, wherein the magnetic material is iron oxide, preferably magnetite.

21. The core/shell particle as claimed in claim 18, wherein the inorganic material is a nanoscale oxide powder.

22. The core/shell particle as claimed in claim 18, wherein the inorganic material is titanium oxide (TiO₂).

23. The core/shell particle as claimed in claim 12, wherein the nanoscale particles which form the core have a particle size between 5 nm and 50 nm, preferably between 5 nm and 20 nm.

24. The core/shell particle as claimed in claim 12, wherein the core/shell particles have a particle size between 5 nm and 100 nm, preferably between 10 nm and 50 nm, in particular between 20 nm and 45 nm.

25. The core/shell particle as claimed in claim 12, wherein they are applied to an inorganic or organic carrier or incorporated into an inorganic or organic matrix.

26. The use of the core/shell particles as claimed in claim 12 as biocides.

27. The use of the core/shell particles as claimed in claim 18 for wastewater treatment, in particular for removing heavy metals from wastewaters.
